(19) United States
(12) Patent Application Publication
(10) Pub. No.: US 2023/0095227 A1
(43) Pub. Date: Mar. 30, 2023

(54) METHOD OF DETERMINING THE BLOOD PRESSURE OF A USER WITHOUT USING A CUFF

(71) Applicant: Commissariat à l'Energie Atomique et aux Energies Alternatives, Paris (FR)

(72) Inventors: Rémi GERBELOT BARILLON, Grenoble Cedex 09 (FR); Pierre BLANDIN, Grenoble Cedex 09 (FR); Stéphane BONNET, Grenoble Cedex 09 (FR); Patrice CAILLAT, Grenoble Cedex 09 (FR)

(73) Assignee: Commissariat à l'Energie Atomique et aux Energies Alternatives, Paris (FR)

(21) Appl. No.: 17/937,055

(22) Filed: Sep. 30, 2022

(30) Foreign Application Priority Data
Sep. 30, 2021 (FR) 21 10386

(51) Int. Cl. A61B 5/021; A61B 8/08; A61B 8/00; A61B 5/024

(52) U.S. Cl. CPC A61B 5/02125; A61B 8/0891; A61B 8/4416; A61B 5/02433; A61B 8/5261; A61B 8/5223; A61B 8/4209; A61B 2562/066; A61B 2562/0242

(57) ABSTRACT

A device is for estimating a blood pressure of a user by a combination of an acoustic mode employing acoustic emitters and acoustic detectors and an optical mode using light sources and photodetectors forming source—detector pairs. The device includes an acoustic selection unit to determine the pertinent acoustic emitter—acoustic detector pair as well as an optical selection unit to determine the pertinent light source—photodetector pairs.

### TECHNICAL FIELD

The technical field is the characterisation of blood pressure without compression.

### PRIOR ART

Most devices enabling characterisation of blood pressure use a pressure sensor coupled to a compression cuff disposed on a limb, generally an arm. The blood pressure is characterised by measuring the pressure exerted by the cuff at one or more characteristic times. The pressure sensor or the acoustic sensor is sensitive to the beating of the heart and to the amplitude thereof.

The devices used by medical personnel (auscultatory method) consist of a cuff the pressure of which is monitored and read by the doctor and generally associated with a stethoscope. When the cuff is deflated, the appearance and the disappearance of sounds known as Korotkoff sounds are detected. The pressure applied by the cuff at the time of the appearance and the disappearance of the sounds corresponds to the systolic pressure and to the diastolic pressure, respectively.

In consumer sphygmomanometers a pressure sensor determines the air pressure in the cuff. The cuff is compressed beforehand in such a manner as to obtain an arterial occlusion. Upon deflation of the cuff pressure oscillations occur. The oscillations increase until a transient maximum amplitude is reached. At this moment the pressure in the cuff is considered equal to the mean blood pressure. On the basis of the detected maximum amplitude, the systolic and diastolic blood pressures are estimated on the basis of empirical laws.

However, if it is wished to measure the pressure continuously use of a device including a cuff presupposes regular compression phases. This constitutes a source of discomfort linked both to the perception of the compression and to the noise of the pump activating the compression of the cuff. Moreover, repeated occlusion at too high a frequency may represent a risk.

Recent developments have lead to being able to effect so-called “cuffless” blood pressure measurements. The publication [1] Nabeel M. “Bi-modal arterial compliance probe for calibration-free cuffless blood pressure estimation”, IEEE transactions on biomedical engineering, Vol. 5, No. 11, November 2018, describes a device coupling an acoustic mode and an optical mode to estimate the blood pressure of a user without recourse to compression of a limb of the user by means of a cuff. The acoustic mode enables measurement of the evolution of the diameter of the artery between two extreme values respectively corresponding to systole and diastole. The optical mode, in accordance with the principles of PPG (infrared photo-plethysmography) enables estimation of a pulse wave velocity (PWV) between two measurement points at a distance from one another along the artery. The pulse wave velocity is usually designated by the abbreviation PWV. The measurements resulting from the two methods are combined so as to be able to estimate the blood pressure.

The principles disclosed in publication [1] may be used to design a device worn by a user and enabling continuous monitoring of the blood pressure, reducing the discomfort felt by the user. However, the measurements effected using the two modes may be affected by uncertainties linked to the positioning of the active components (light sources, acoustic or optical sensors) relative to the artery. Thus when the device is applied to the body of a user the sensors must be disposed correctly and precisely relative to the artery so that the variation of the diameter and the pulse wave velocity are estimated correctly.

Also, the device described above presupposes precise positioning on the skin of the user. Another difficulty is linked to movements of the user, which may lead to variation of the position of the sensors relative to the artery. The invention described hereinafter enables these difficulties to be overcome.

### SUMMARY OF THE INVENTION

A first object of the invention is a device for estimation of a blood pressure of a user, the device being intended to be worn by the user, the device including:
a support intended to be applied against the skin of the user;
a plurality of light sources disposed on the support and configured to emit light toward the skin of the user when they are activated;
a plurality of photodetectors disposed on the support at a distance from each light source and configured to detect light emanating from the skin of the user following activation of at least one light source, each photodetector forming with said light source a source—photodetector pair;

the device being characterised in that it includes:
a plurality of acoustic transducers, including at least:
   an acoustic emitter configured to emit an acoustic wave through the skin; and
   an acoustic detector configured to detect an acoustic wave reflected in the body of the user and propagating through the skin;
an acoustic selection unit programmed:
   to take into account an acoustic selection criterion;
   in accordance with the acoustic selection criterion, to select an acoustic emitter and an acoustic detector from among the acoustic transducers, the selection being effected as a function of an acoustic signal detected by each acoustic detector following emission of an acoustic wave by at least one acoustic emitter;
an optical selection unit configured:
   to take into account an optical selection criterion;
   in accordance with the optical selection criterion, to select a first light source—photodetector pair including a first light source and a first photodetector chosen from among the light sources and the photodetectors and a second light source—photodetector pair including a second light source and a second photodetector chosen from among the light sources and the photodetectors, the selection being effected as a function of the signals detected by the first photodetector and the second photodetector following activation of the first light source and of the second light source;
a central unit programmed to estimate a blood pressure from:
   the signal detected by the selected acoustic detector;
   the signals detected by the first photodetector and the second photodetector.

The central unit may be programmed:
to estimate an arterial diameter from the signal detected by the selected acoustic detector;
to estimate a pulse wave velocity from the signals detected by the first photodetector and the second photodetector;
to estimate the blood pressure as a function of the estimated arterial diameter and the estimated pulse wave velocity.

The light source preferably emits light in a spectral band between 500 nm and 1200 nm inclusive.

The device may include:
a first group of light sources and of photodetectors;
a second group of light sources and of photodetectors at a distance from the first group of light sources and of photodetectors.

The optical selection unit is configured:
to select the first light source and the first photodetector from among the light sources and the photodetectors of the first group;
to select the second light source and the second photodetector from among the light sources and the photodetectors of the second group.

In accordance with one possibility, the acoustic selection criterion being a signal-to-noise ratio, the acoustic selection unit is configured:
to estimate a signal-to-noise ratio of each signal detected by an acoustic detector;
to select the acoustic detector for which the signal-to-noise ratio is the highest.

The acoustic selection criterion may be an intensity of the signal detected by an acoustic detector. The acoustic selection unit is then configured
to estimate an intensity of each signal detected by an acoustic detector;
to select the acoustic detector the intensity of which is the highest.

In accordance with one possibility, the optical criterion being a correlation criterion, the optical selection unit is configured:
to estimate a temporal correlation between the signals detected at different times by photodetectors of each light source—photodetector pair;
to select the first light source and the first photodetector as well as the second light source and the second photodetector as a function of the estimated temporal correlation.

In accordance with one possibility, the optical criterion is an amplitude criterion. The optical selection unit is configured:
to estimate an amplitude of a temporal evolution of signals detected at various times by photodetectors of each light source—photodetector pair;
to select the first light source and the first photodetector as well as the second light source and the second photodetector as a function of that amplitude.

In accordance with one possibility, the optical criterion is a form criterion. The optical selection unit is configured:
to take into account a predetermined temporal form;
to determine a temporal evolution of the signals detected at different times by the photodetectors of each light source—photodetector pair;
to select the first light source and the first photodetector as well as the second light source and the second photodetector as a function of a correlation between the temporal evolution of the signals detected and the predetermined temporal form.

Another object of the invention is a method of estimation of a blood pressure using a device conforming to the first object of the invention, the method including:
a) disposing the support on the skin of a user, facing an artery;
b) emitting at least one incident acoustic wave by means of an acoustic emitter and acquiring acoustic signals by means of an acoustic detector, each acoustic signal detected including echoes representative of reflections of the incident acoustic wave by the artery, the step b) being carried out for different acoustic emitters and/or different acoustic detectors so that each acoustic signal detected is associated with an acoustic emitter and an acoustic detector;
c) using the acoustic selection unit:
   taking into account an acoustic selection criterion;
   selecting an acoustic emitter and an acoustic detector as a function of a confrontation between each acoustic signal detected during the step b) and the acoustic selection criterion;
d) for each light source, emitting incident light toward the skin of the user and detecting back-scattered radiation by means of at least one photodetector, each photodetector generating an optical signal representative of the intensity of the back-scattered radiation;
e) using the optical selection unit:
   taking into account an optical selection criterion;
   selecting two light source—photodetector pairs, each pair including a light source and a photodetector, as a function of a confrontation between each optical signal from each photodetector and the optical selection criterion;
f) emitting an incident acoustic wave from the acoustic transducer selected in c) and forming an acoustic signal representative of echoes following reflection of the incident acoustic wave by the artery;
g) activating light sources of each light source—photodetector pair selected in e) and each photodetector of each pair forming an optical signal representative of the intensity of the radiation back-scattered by the artery;
h) as a function of the acoustic signal and of the optical signals formed by each photodetector at different times, estimating the blood pressure of the user.

In accordance with one possibility, the step h) includes:
h1) estimating the diameter of the artery as a function of the formed acoustic signal;
h2) estimating a pulse wave velocity as a function of the optical signals formed at different times by each selected photodetector;
h3) estimating a blood pressure of the user from the resulting diameter of the artery from the sub-step h1) and from the resulting pulse wave velocity from the substep h2).

The substep h2) may include estimating a temporal offset between the optical signals respectively formed by the first photodetector and the second photodetector.

The steps a) to e) may constitute a phase of calibration of the device, the steps f) to h) being reiterated between two successive calibrations.

In accordance with one possibility, the device includes:
a first group of light sources and of photodetectors;
a second group of light sources and of photodetectors distant from the first group of light sources and of photodetectors.

The method may then include:
selecting a first light source—photodetector pair in the first group;
selecting a second light source—photodetector pair in the second group.

In accordance with one embodiment, the method includes:
taking into account a range of validity of the blood pressure;
renewing the calibration phase if the resulting blood pressure from the step h) is situated outside the range of validity.

In accordance with one possibility, the acoustic selection criterion is a maximum signal-to-noise ratio or a maximum intensity of a detected acoustic signal, the selection of the acoustic emitter and of the acoustic detector being effected as a function of the acoustic signal associated with the acoustic emitter—acoustic detector pair the signal-to-noise ratio of which is the maximum.

The optical selection criterion may include a temporal correlation criterion, ignoring a temporal offset, so that the selection of each source—detector pair includes:
estimating a temporal correlation between the signals detected by the photodetectors of each light source—photodetector pair at different times;
determining the light source—photodetector pairs for which the resulting signal from the photodetector has the highest temporal correlation.

The optical selection criterion may be one of the selection criteria described in connection with the first object of the invention.

The method may be such that:
the first light source—photodetector pair defines a first measurement point;
the second light source—photodetector pair defines a second measurement point;
the first measurement point and the second measurement point are at a distance from one another.

The invention will be better understood on reading the description of embodiments given in the remainder of the description with reference to the figures listed below.

### FIGURES

FIG. 1 is an example of a device in accordance with the invention.

FIGS. 2A to 2E illustrate one embodiment of the acoustic mode of the device.

FIGS. 3A and 3B illustrate one embodiment of the optical mode of the device.

FIG. 4 is another example of a device in accordance with the invention.

FIG. 5 represents diagrammatically the main steps implementing a method of determining a blood pressure of a user using a device as described in connection with FIG. 1 or FIG. 4.

### DESCRIPTION OF PARTICULAR EMBODIMENTS

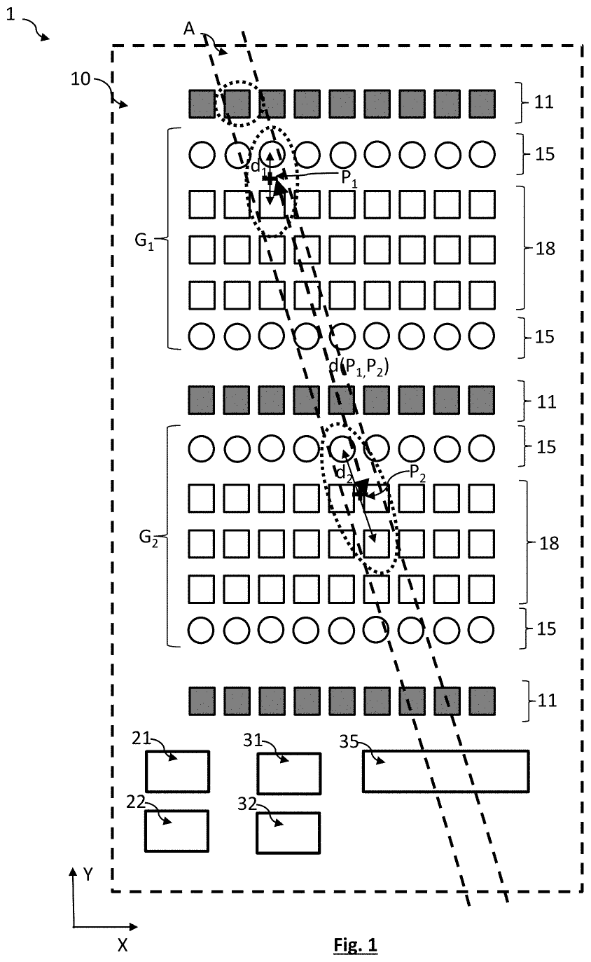

FIG. 1 shows an example of a device 1 enabling estimation of the blood pressure of a user. The device 1 includes a support 10 intended to be placed against the skin of a user facing an artery A. This may for example be the carotid artery as described in [1] or the radial artery in the case of a wrist device. The support 10 is configured to be placed durably in contact with the skin. It is preferably a flexible support so as to espouse the contour of the skin. The support 10 may be adhesive or connected to a means of retention against the skin, of cuff or elastic bracelet type.

The support includes components enabling use of acoustic or optical modes as described in [1].

The acoustic mode uses acoustic transducers 11 distributed on the support.

Each acoustic transducer is configured to emit and/or to detect an ultrasound acoustic wave so as to determine a temporal evolution D(t) of the diameter of the artery A because of the effect of cardiac activity. The acoustic transducers may be piezoelectric transducers or electromechanical sensors of MEMS type. The acoustic transducers are connected to an acoustic processing unit 31. Although this is not necessary, each acoustic transducer may preferably function both as an acoustic emitter and an acoustic detector.

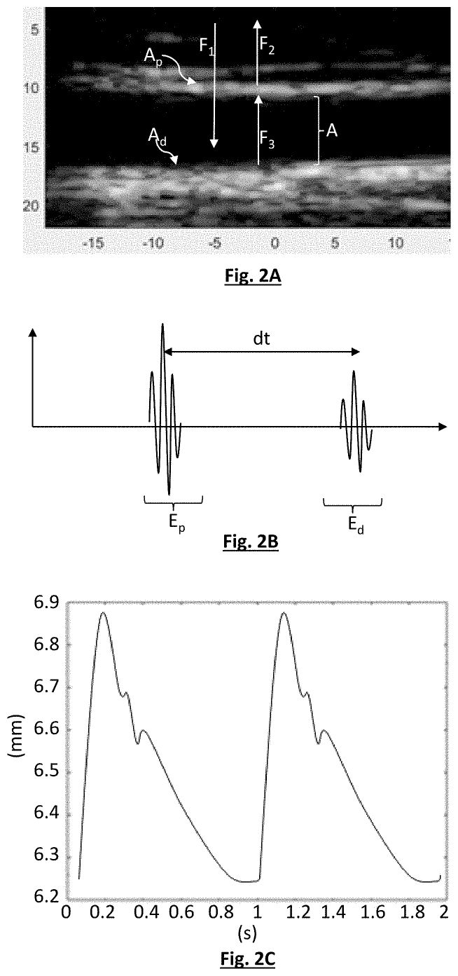

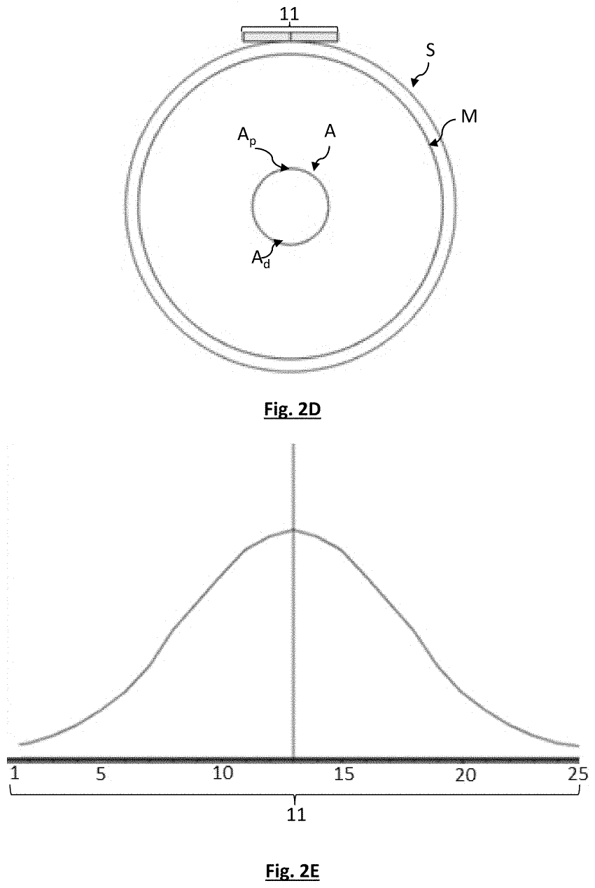

The device includes an acoustic processing unit 31 configured to receive signals detected by at least one acoustic transducer 11 functioning as an acoustic detector so as to estimate the diameter D(t) of the artery at different times. The acoustic mode is described in more detail with reference to FIGS. 2A to 2E.

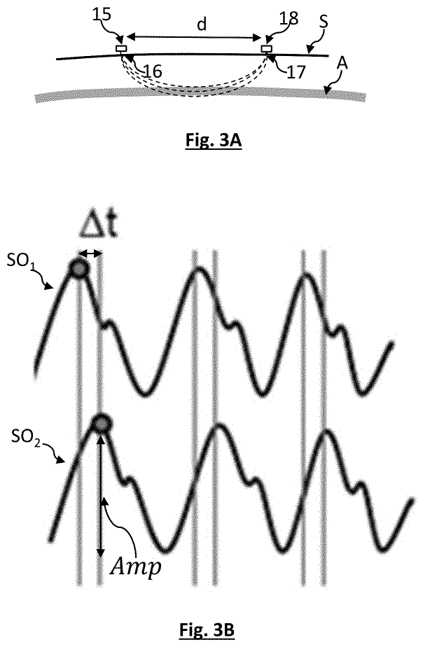

The optical mode employs light sources 15 and photodetectors 18 distributed on the support 10 at a distance from one another. Each light source is configured to emit light toward the skin of the user when it is activated. Each photodetector is at a distance from a light source. Each photodetector is configured to detect light emanating from the skin of the user following activation of at least one light source. Each photodetector is therefore able to detect light emitted by a light source and propagating through the body of the user before emerging from the skin of the user opposite the photodetector. When the photons detected by a photodetector propagate between the skin and the artery the light detected undergoes periodic variations because of the effect of the periodic variation of the blood volume induced by cardiac activity in the probed tissues (arteries, but also veins, capillaries, . . . ). The optical mode is described in more detail with reference to FIGS. 3A and 3B.

As a general rule the optical mode assumes the taking into account of the light source—photodetector pairs, each pair combining a light source and a photodetector at a distance from the light source. The distance between a light source and a photodetector of the same pair may be of the order of a few millimetres to a few cm. A photodetector (respectively a light source) may form different pairs with different light sources (respectively different photodetectors).

The photodetectors are connected to an optical processing unit 32. The optical processing unit 32 is configured to receive signals detected by at least one photodetector 18 so as to estimate at different times the time taken by the disturbance of the blood volume in the tissues induced by cardiac activity to propagate between two measurement points P₁, P₂ spaced by a separation distance A along an arterial segment. Each measurement point is situated between a light source and a photodetector. The pulse wave velocity PWV is estimated on the basis of a ratio between:
a temporal offset Δt between the respective signals detected by each photodetector 18;
the separation distance d(P₁, P₂) between the two measurement points P₁ and P₂.

The support 10 is placed on the skin of the user facing the analysed artery A. However, the position of the artery relative to the support 10 is not known precisely, in particular if the user is liable to move. An important aspect of the invention is the ability to select:
a pertinent acoustic emitter and a pertinent acoustic detector, that is to say ones correctly positioned relative to the artery A, so as to obtain a correct estimate of the temporal variation of the diameter of the artery;
pertinent light source—photodetector pairs, each pair including a light source 15 and a photodetector 18, each pair being positioned so as to determine a variation of the blood volume at the two measurement points P₁ and P₂. Comparison of the signals from each photodetector enables estimation of the pulse wave velocity.

The device includes an acoustic selection unit 21 connected to each acoustic detector. The acoustic selection unit 21 is programmed to take into account an acoustic selection criterion and to select an acoustic emitter and acoustic detector from among the acoustic transducers. The acoustic emitter and the acoustic detector selected are those for which, following the emission of an acoustic wave by the acoustic emitter, the acoustic detector detects an acoustic signal considered as best satisfying the acoustic selection criterion. The acoustic selection criterion is for example a maximum signal-to-noise ratio. In this case the acoustic detector selected by the acoustic selection unit is for example that which during a measurement period generates the acoustic signal with the highest signal-to-noise ratio. In FIG. 1 a selected acoustic transducer functioning both as an acoustic emitter and as an acoustic detector is diagrammatically represented in dashed outline. The selected acoustic transducer is centred (or considered such) relative to the artery A. The resulting signal from the selected transducer is then transmitted to the acoustic processing unit 31 in order to determine the temporal evolution D(t) of the diameter of the artery.

The device includes an optical selection unit 22 connected to each photodetector 18. The optical selection unit 22 is programmed to take into account an optical selection criterion and to select two resulting optical signals from the respective photodetectors of two light source—photodetector pairs. The selected pairs are those for which the photodetectors generate an optical signal considered as best satisfying the optical selection criterion. The optical selection criterion is for example a correlation between the respective signals generated by the photodetector of each pair. In this case the light source—photodetector pairs selected by the optical selection unit 22 are those generating optical signals that are temporally correlated, ignoring the temporal offset. By temporally correlated is meant that the evolutions as a function of time of the respective signals at the two measurement points are correlated. The temporal offset depends on the distance between respective measurement points defined by each light source—photodetector pair. In FIG. 1 two selected light source—photodetector pairs are diagrammatically represented in dashed line. It is considered that each of the measurement points P₁ and P₂ is situated facing the middle of a straight line segment connecting the light source to the photodetector of the selected light source—photodetector pair. In FIG. 1 there have been represented:
a double-headed arrow indicating a first, so-called back-scattering distance d₁ between the source and the photodetector of the first selected light source—photodetector pair;
a double-headed arrow indicating a second back-scattering distance d₂ between the source and the photodetector of the second selected light source—photodetector pair.

Each selected light source—photodetector pair preferably extends to either side of the artery or along the artery.

The respective measurement points P₁ and P₂ formed by the two selected light source—photodetector pairs are spaced from one another. The distance d(P₁, P₂) along the artery between the two measurement points is preferably greater than 1 cm or than 5 cm.

The resulting signals from the photodetectors of each selected light source—photodetector pair are sent to the optical processing unit 32 in order to estimate the pulse wave velocity PWV.

The light sources 15 and the photodetectors 18 are preferably distributed on the support 10 to form a first group G₁ and a second group G₂. The optical selection unit 22 is programmed to select:
a first light source—photodetector pair from among the light sources 15 and the photodetectors 18 of the first group G₁;
a second light source—photodetector pair from among the light sources 15 and the photodetectors 18 of the second group G₂.

This distribution in two groups makes it possible to guarantee a minimum separation distance d(P₁, P₂) between the respective first and second measurement points P₁, P₂ defined by each selected light source—photodetector pair. The distance d(P₁, P₂) between the measurement points P₁ and P₂ is assumed known by virtue of the known geometry of the device. It is represented in FIG. 1 by a dashed line double-headed arrow.

FIGS. 2A, 2B and 2C illustrate the operation of the acoustic mode. Each transducer emits an incident acoustic wave symbolically represented by the arrow F₁. The latter wave propagates through the skin of the user toward the artery A. A portion of the incident acoustic wave is successively reflected by a proximal portion Aₚ and a distal portion A_{d} of the wall of the artery A that are diametrically opposite. In FIG. 2A the arrows F₂ and F₃ respectively correspond to the respective portions of the incident wave reflected by the proximal portion Aₚ and the distal portion A_{d}. The transducer (or another transducer) detects the waves successively reflected by the proximal portion Aₚ and the distal portion A_{d}, the latter respectively forming a proximate echo Eₚ and a distal echo Ed. FIG. 2B diagrammatically represents the echoes. In FIG. 2B the ordinate axis corresponds to the amplitude of the detected wave and the abscissa axis corresponds to time. The temporal offset dt between the two echoes Eₚ and Ed enables estimation of the diameter of the artery, given the known propagation velocity of the acoustic wave. The measurements represented in FIG. 2B diagrammatically represent an acoustic signal sent by each transducer 11 to the selection unit 21.

The measurements diagrammatically represented in FIG. 2B are repeated at different measurement times at a sampling frequency that may be 1 kHz for example. FIG. 2C represents a temporal variation D(t) of the diameter of the artery (ordinate axis—units μm) as a function of time t (units:second). The maximum diameter corresponds to the diameter at systole while the minimum diameter corresponds to the diameter at diastole. In this example the variation ΔD of diameter between the diameters at systole and at diastole is of the order of 0.6 mm.

The position of the transducer 11 relative to the artery impacts the quality of the measurement of D(t). Simulations have shown that if the transducer is not centred relative to the artery the intensity of the reflected acoustic wave is reduced. Given measurement noise, this increases the uncertainty in the determination of the temporal interval dt and therefore of the temporal evolution D(t). FIG. 2D shows a simulation configuration. There has been simulated an acoustic probe including a row of 25 transducers 11 each functioning in emitter/detector mode disposed in contact with skin S of cylindrical shape and having a radius approximately 13 mm. The artery A has been modelled at the centre of the cylinder modelling the skin S. Between the skin and the artery extends a medium M corresponding to a muscle. The artery A is therefore situated at a depth below the skin S between 10 mm and 15 mm inclusive, which corresponds to a normal artery depth. The intensity of the echoes produced by each transducer in response to an incident acoustic wave has been evaluated using the CIVA software (from Extende). The densities and velocities of acoustic propagation in the modelled media S, M and A are representative of those of blood, muscle and skin, respectively. FIG. 2E represents the evolution of the amplitude of the signal (Y axis—arbitrary units) as a function of the transducer (X axis). In this case emission and reception are performed by the same transducer. The transducers 1, 13 and 25 respectively correspond to the extreme left, centred and extreme right positions of the probe. It is seen that a centred position of the acoustic transducer relative to the artery enables a maximum echo intensity to be obtained. Thus the position of the transducer has an important influence on the quality of the measurement effected. The acoustic selection unit 21 enables selection of the acoustic transducer generating the echoes of which the signal is the maximum from among those from the other transducers. A result of this is an accurate estimate of the temporal evolution D(t) of the diameter D of the artery.

In accordance with one possibility transducers phase-shifted relative to one another are used simultaneously. The selection unit enables selection of a reference transducer followed by estimation of a temporal offset between the various transducers relative to the reference transducer.

FIGS. 3A and 3B illustrate the operation of the optical mode. There have been represented a light source 15 and a photodetector 18 forming a light source—photodetector pair. The light source 15 emits an incident light beam 16 propagating through the skin toward the artery A of the user. The emission direction is generally perpendicular to the skin. The light beam is preferably emitted in a narrow emission spectral band, preferably <50 nm, in a spectral range between 500 nm and 1200 nm inclusive, and preferably between 500 nm and 1000 nm. This spectral range corresponds to significant absorption of light by haemoglobin. The spectral bands may be centred on the following wavelengths: 525 nm, 660 nm, 740 nm, 805 nm, 850 nm.

Each light source may be a LED (light-emitting diode) or an end of an optical fibre the other end of which is disposed facing an illumination source. Each photodetector may be a photodiode or an end of an optical fibre the other end of which is connected to a light sensor. Alternatively, each light source may be a laser diode, a VECSEL or the end of a light guide, for example an optical fibre.

The photons of the incident light beam 16 penetrate into the body of the user. They propagate in the tissues between the skin and the artery A, the latter being situated at a depth below the skin of the order of 10 mm. Some of the incident photons are back-scattered in a direction parallel to the emission direction. The back-scattered photons constitute back-scattered radiation 17. The back-scattered radiation 17 can be detected by the photodetector 18 placed facing the skin of the user. The distance d between the light source and the photodetector, termed the back-scattering distance, is generally non-zero and is generally between 5 mm and a few cm inclusive. The photodetector 18 thus enables measurement of the intensity of the radiation back-scattered over the back-scattering distance d. In FIG. 3A the curved dashed line arrows represent optical paths of photons emitted by the light source 15 and detected by the photodetector 18. The photodetector is therefore adapted to measure an intensity of a light beam formed by back-scattered photons.

The greater the back-scattering distance the greater the depth the photons constituting the back-scattered radiation 17 penetrate the tissues of the user. The intensity of the back-scattered radiation depends on the variation of blood volume in the artery. The greater the quantity of blood the greater the quantity of photons absorbed by haemoglobin and the lower the intensity of the back-scattered radiation. On each heart beat the afflux of blood causes in the probed tissues modulation of the absorption of the light propagating in the tissues. A result of this is modulation of the intensity of the back-scattered radiation detected by the photodetector 18 associated with the light source.

The signal detected by the photodetector 18 includes a continuous component to which is added a pulsed component, the latter varying as a function of cardiac activity. The intensity detected by the photodetector therefore includes a periodic component the fundamental frequency of which corresponds to the cardiac frequency.

As represented in FIG. 1, the device enables selection of two light source—photodetector pairs respectively defining two measurement points P₁ and P₂ along the artery spaced by a separation distance d(P₁, P₂). Because of the separation distance, the respective periodic resulting signals from the first and the second light source—photodetector pairs are offset by a temporal offset Δt. Knowing the separation distance d(P₁, P₂) the estimate of the temporal offset, termed the transit time of the pulse wave, enables estimation of a velocity termed the “pulse wave velocity” (PWV) using the following expression:

$$P W V = \frac{d ( {P}_{1} , {P}_{2} )}{Δ t} \qquad ( 1 )$$

FIG. 3B represents:
a first resulting optical signal SO₁ from the photodetector of the first light source—photodetector pair;
a second resulting optical signal SO₂ from the photodetector of the second light source—photodetector pair.

In FIG. 3B each signal corresponds to the opposite of the detected signal: thus each signal SO₁, SO₂ is representative of the periodic absorption by the tissues at the measurement points P₁ and P₂. The temporal offset can be estimated on the basis of the temporal difference Δt between notable points on each curve, for example maxima or minima.

The separation distance d(P₁, P₂) is preferably greater than 5 mm. It is preferably less than 10 cm or 5 cm.

The frequency of acquisition of the resulting signals from each photodetector may be between 100 Hz and 100 kHz inclusive, which enables estimation of the pulse wave velocity with sufficient temporal resolution.

However, the depth of the artery below the skin is not known precisely. Similarly, the position of the artery parallel to the support 10 is not known or may vary as a function of movements of the user. It is therefore difficult to determine a priori the most pertinent source—photodetector pairs for accurate estimation of the pulse wave velocity. It is clear in FIG. 3A that the back-scattering distance must be sufficient to enable propagation of the detected photons through the artery or a volume of tissue impacted by the perturbation of the blood volume induced by cardiac activity. It is also preferable for the source and the photodetector of the same pair to be disposed either along the artery or on either side of the artery so that the measurement point situated between the source and the photodetector is located close to the artery.

As represented in FIG. 3B the optical selection unit 22 takes into account various detected signals. These are optical signals representative of the intensity detected by the photodetectors of different light source—photodetector pairs. The detected signals include a so-called pulsed component that can be considered periodic because of the repetition of the cardiac cycle. The pertinent source—photodetector pair is selected taking into account an optical selection criterion and determining two light source—photodetector pairs for which the optical selection criterion is satisfied.

The optical selection criterion may be a temporal correlation. In fact, the most pertinent detected signals have a comparable temporal evolution that corresponds to cardiac activity. The fact of selecting detected signals having a high temporal correlation facilitates determination of the temporal offset Δt illustrated in FIG. 3B. By temporal correlation is meant correlation ignoring a temporal offset that corresponds to the distance between the measurement points along the artery.

Other optical selection criteria may be applied in addition to or instead of the correlation criterion. During each period the pulsed component of each detected signal describes an oscillation of a certain amplitude Amp. The acceptance criterion may be an amplitude greater than a certain threshold or situated in a predefined acceptance range.

The optical selection criterion may be a correlation of the temporal evolution of each detected signal during a period with a predetermined form. Two light source—photodetector pairs are then selected having the best correlation with a predetermined temporal form.

Another optical selection criterion may be a minimum distance between the source and the photodetector forming a light source—photodetector pair so that the depth addressed is sufficient.

The optical selection criteria described above may be combined.

The optical processing unit 32 then estimates the pulse wave velocity from optical signals generated by the photodetectors of the selected light source—photodetector pairs.

The device includes a calculation unit 35 configured to estimate an arterial pressure value from the resulting temporal evolution D(t) of diameter from the acoustic processing unit 31 and the resulting PMV from the optical processing unit 32. The blood pressure P is such that

P=f(D,PWV) (2)

where f is a predefined function.

For example, the function f may be such that:

$$P ( t ) - {P}_{0} = 2 {ρ ( P W V )}^{2} ln ( \frac{D ( t )}{{D}_{0}} ) \qquad ( 3 )$$

where:
P(t) is the blood pressure at time t,
D₀ is a reference diameter, for example the diameter at end of diastole,
P₀ is a reference pressure corresponding to D=D₀.

The blood pressure P(t) obtained from equation (3) describes a periodic function, each period corresponding to one heartbeat. In each period the maximum pressure corresponds to the systolic blood pressure and the minimum pressure corresponds to the diastolic blood pressure. The mean blood pressure is the mean of the pressure over a period.

The function f previously referred to may be determined by calibration in the presence of a reference measurement of the blood pressure of the user. It is then possible to establish a link between the values (D, PWV) measured at various times with a reference blood pressure measured by the reference method. The confrontation between the measured values and the measured reference pressure enables the calibration function to be established.

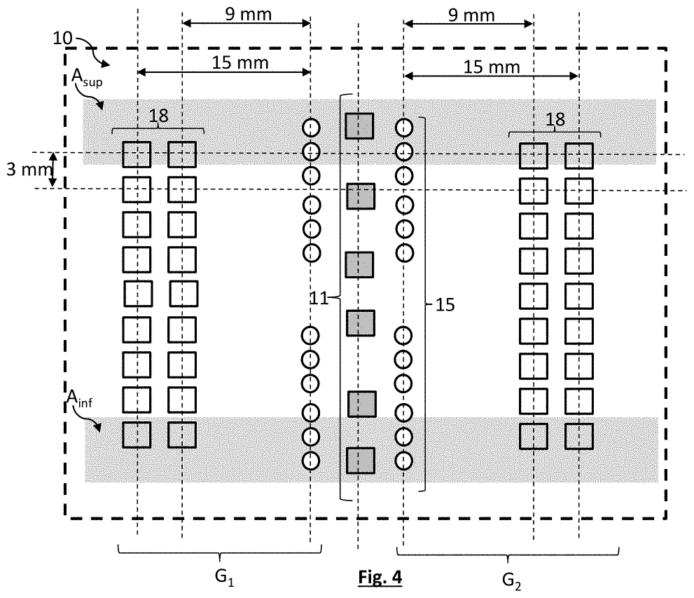

FIG. 4 represents another example of a device suitable for implementing the invention. The main dimensions have been indicated in FIG. 4. The device enables the analysis of an artery A extending between the two extreme positions Aₛᵤₚ and A_{inf} represented in FIG. 4. The two groups G₁ and G₂ in which the two light source—photodetector pairs are to be identified as a function of the position of the artery relative to the support 10 are represented by an accolade bracket.

In a variant, the support 10 is divided into two elementary supports at a distance from one another. The first elementary support includes a first group G₁ of photodetectors and of light sources. A second elementary support includes a second group of photodetectors and of light sources. The transducers may be fixed to one of the elementary supports or to the two elementary supports. The support 10 then need not be monolithic and may include different elementary supports.

The acoustic or optical selection units and the central unit may form one unit implemented by a microprocessor. Alternatively, each of these units uses a microprocessor. In accordance with one possibility, some or all of the selection units or the central unit is or are remotely sited, at a distance from the support 10. The device includes a transmission unit so as to transmit the signals from the transducers and the photodetectors over a wired or wireless connection.

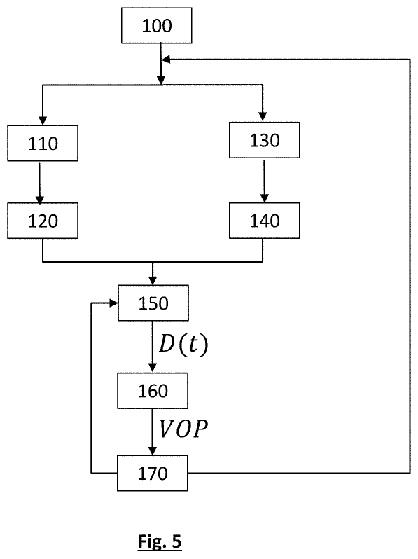

FIG. 5 diagrammatically represents the main steps of a method employing the device as represented in FIG. 1 or 4.

Step 100: placing the support on the skin of a user, facing an artery.

Step 110: an acoustic emitter emitting an acoustic wave and an acoustic detector detecting a reflected acoustic wave, the step 110 being repeated for different acoustic emitters and/or different acoustic detectors. Each detected acoustic signal is liable to include echoes representative of the reflection by the artery of the acoustic wave emitted by the acoustic emitter.

Step 120: the acoustic selection unit processing the acoustic signals so as to identify the pertinent acoustic emitter—acoustic detector pair. The latter corresponds to the detector from which the acoustic signal detected following emission of an acoustic wave by the acoustic emitter best satisfies the acoustic selection criterion referred to above.

Step 130: various photodetectors of various light source—photodetector pairs acquiring optical signals. During this step a light source may be activated sequentially and optical signals acquired from different photodetectors considered sufficiently close to the source to detect usable back-scattered radiation. The light sources are activated successively. Optical signals are then obtained for each light source—photodetector pair.

Step 140: the optical selection unit processing the resulting optical signals from each photodetector during the step 130 so as to identify the pertinent light source—photodetector pairs. The latter correspond to the pairs for which the resulting signals from the photodetector best satisfy the optical selection criterion.

Steps 110 to 140 correspond to a calibration phase based on calibration measurements acquired during steps 110 and 130. Calibration measurements may be acquired during a predefined calibration period, for example a few minutes.

Steps 150 to 170 correspond to the use of the device following the calibration phase.

Step 150: determining the temporal evolution of the diameter D(t) of the artery using the acoustic mode. During this step the acoustic processing unit 31 receives the resulting acoustic signal from the acoustic detector selected in the step 120.

Step 160: determining the evolution of the pulse wave velocity PWV of the artery using the optical mode. During this step the optical processing unit 32 receives the resulting optical signals from the respective photodetectors of the two light source—photodetector pairs selected in the step 140.

The steps 150 and 160 may be effected simultaneously or in either order.

Step 170: estimating the blood pressure as a function of the resulting diameter D(t) of the artery and of the resulting PWV from the steps 150 and 160, for example using the expression (3) or another calibration function.

The steps 150 to 170 may be reiterated at successive measurement times so as to effect a “continuous” measurement of the blood pressure, that is to say one at sufficiently close together times. The calibration phase (steps 110 to 140) may be reiterated periodically so as to identify the most pertinent acoustic transducers and light source—photodetector pairs. The calibration phase may equally be effected in the event of a blood pressure measurement considered abnormal occurring.

The invention benefits from the fact that the device enables estimation of a blood pressure without recourse to compression of a limb of the user. It is robust with regard to uncertainties of the positioning of the support of the device on the skin of the user as well as any movement of the user wearing the support.

1. A device for estimation of a blood pressure of a user, the device being configured to be worn by the user, the device comprising:
a support configured to be applied against the skin of the user;
a plurality of light sources disposed on the support and configured to emit light toward the skin of the user when they are activated;
a plurality of photodetectors disposed on the support at a distance from each light source and configured to detect light emanating from the skin of the user following activation of at least one light source, each photodetector forming with said light source a source—photodetector pair;
a plurality of acoustic transducers, including at least:
an acoustic emitter configured to emit an acoustic wave through the skin; and
an acoustic detector configured to detect an acoustic wave reflected in the body of the user and propagating through the skin;
an acoustic selection unit programmed:
to take into account an acoustic selection criterion;
in accordance with the acoustic selection criterion, to select an acoustic emitter and an acoustic detector from among the acoustic transducers, the selection being effected as a function of an acoustic signal detected by each acoustic detector following emission of an acoustic wave by at least one acoustic emitter;
an optical selection unit configured:
to take into account an optical selection criterion;
in accordance with the optical selection criterion, to select a first light source—photodetector pair including a first light source and a first photodetector chosen from among the light sources and the photodetectors and a second light source—photodetector pair including a second light source and a second photodetector chosen from among the light sources and the photodetectors, the selection being effected as a function of the signals detected by the first photodetector and the second photodetector following activation of the first light source and of the second light source; and
a central unit programmed to estimate a blood pressure from:
the signal detected by the selected acoustic detector;
the signals detected by the first photodetector and the second photodetector.

2. The device according to claim 1, wherein the central unit is programmed:
to estimate an arterial diameter (D(t)) from the signal detected by the selected acoustic detector;
to estimate a pulse wave velocity (PWV) from the signals detected by the first photodetector and the second photodetector;
to estimate the blood pressure (P(t)) as a function of the estimated arterial diameter and the estimated pulse wave velocity.

3. The device according to claim 1, wherein each light source emits light in a spectral band between 500 nm and 1200 nm inclusive.

4. The device according to claim 1, comprising:
a first group of light sources and of photodetectors;
a second group of light sources and of photodetectors at a distance from the first group of light sources and of photodetectors;
the optical selection unit is then configured
to select the first light source and the first photodetector from among the light sources and the photodetectors of the first group;
to select the second light source and the second photodetector from among the light sources and the photodetectors of the second group.

5. The device according to claim 1, wherein, the acoustic selection criterion being a signal-to-noise ratio, the acoustic selection unit is configured:
to estimate a signal-to-noise ratio of each signal detected by an acoustic detector;
to select the acoustic detector for which the signal-to-noise ratio is the highest.

6. The device according to claim 1, wherein, the optical selection criterion being a correlation criterion, the optical selection unit is configured:
to estimate a temporal correlation between the signals detected at different times by photodetectors of each light source—photodetector pair;
to select the first light source and the first photodetector as well as the second light source and the second photodetector as a function of the estimated temporal correlation.

7. The device according to claim 1, wherein the optical selection criterion being an amplitude criterion, the optical selection unit is configured:
to estimate an amplitude of a temporal evolution of signals detected at various times by photodetectors of each light source—photodetector pair;
to select the first light source and the first photodetector as well as the second light source and the second photodetector as a function of the amplitude.

8. The device according to claim 1, wherein the optical selection criterion being a form criterion, the optical selection unit is configured:
to take into account a predetermined temporal form;
to determine a temporal evolution of the signals detected at different times by the photodetectors of each light source—photodetector pair;
to select the first light source and the first photodetector as well as the second light source and the second photodetector as a function of a correlation between the temporal evolution of the signals detected and the predetermined temporal form.

9. A method of estimation of a blood pressure using the device according to claim 1, the method comprising:
a) disposing the support on the skin of a user, facing an artery;
b) emitting at least one incident acoustic wave with an acoustic emitter and acquiring acoustic signals with an acoustic detector, each acoustic signal detected including echoes representative of reflections of the incident acoustic wave by the artery, the step b) being carried out for different acoustic emitters and/or different acoustic detectors so that each acoustic signal detected is associated with an acoustic emitter and an acoustic detector;
c) using the acoustic selection unit:
taking into account an acoustic selection criterion;
selecting an acoustic emitter and an acoustic detector as a function of a confrontation between each acoustic signal detected during the step b) and the acoustic selection criterion;
d) for each light source, emitting incident light toward the skin of a user and detecting back-scattered radiation with at least one photodetector, each photodetector generating an optical signal representative of the intensity of the back-scattered radiation;
e) using the optical selection unit:
taking into account an optical selection criterion;
selecting two light source—photodetector pairs, each pair including a light source and a photodetector, as a function of a confrontation between each optical signal from each photodetector and the optical selection criterion;
f) emitting an incident acoustic wave from the acoustic transducer selected in c) and forming an acoustic signal representative of echoes following reflection of the incident acoustic wave by the artery;
g) activating light sources of each light source—photodetector pair selected in e) and each photodetector of each pair forming an optical signal representative of the intensity of the radiation back-scattered by the artery; and
h) estimating the blood pressure of the user as a function of the acoustic signal and of the optical signals formed by each photodetector at different times.

10. The method according to claim 9, wherein the step h) comprises:
h1) estimating the diameter of the artery as a function of the formed acoustic signal;
h2) estimating a pulse wave velocity as a function of the optical signals formed at different times by each selective photodetector;
h3) estimating a blood pressure of the user from the resulting diameter of the artery from the sub-step h1) and from the resulting pulse wave velocity from the substep h2).

11. The method according to claim 10, wherein the substep h2) includes estimating a temporal offset (AO between the optical signals respectively formed by the first photodetector and the second photodetector.

12. The method according to claim 9, wherein the steps a) to e) constitute a phase of calibration of the device, the steps f) to h) being reiterated between two successive calibrations.

13. The method according to claim 9, wherein the device is a device according to claim 4, the method comprising:
selecting a first light source—photodetector pair in the first group;
selecting a second light source—photodetector pair in the second group.

14. The method according to claim 9, comprising:
taking into account a range of validity of the blood pressure;
if the resulting blood pressure from the step h) is situated outside the range of validity, repeating the calibration phase.

15. The method according to claim 9, wherein the acoustic selection criterion is a maximum signal-to-noise ratio, the selection of the acoustic emitter and of the acoustic detector being effected as a function of the acoustic signal associated with the acoustic emitter—acoustic detector pair the signal-to-noise ratio of which is the maximum.

16. The method according to claim 9, wherein the optical selection criterion includes a temporal correlation criterion ignoring a temporal offset so that the selection of each source—detector pair comprises:
estimating a temporal correlation between the signals detected at different times by the photodetectors of each light source—photodetector pair;
determining the light source—photodetector pairs for which the resulting signal from the photodetector has the highest temporal correlation.

17. The method according to claim 9, wherein:
the first light source—photodetector pair defines a first measurement point;
the second light source—photodetector pair defines a second measurement point;
the first measurement point and the second measurement point are at a distance from one another.
